(19) United States
(12) Patent Application Publication
(10) Pub. No.: US 2019/0047933 A1
(43) Pub. Date: Feb. 14, 2019

(54) PROCESS FOR THE SYNTHESIS OF (2E, 4E, 6Z, 8E)-8-(3,4-DIHYDRONAPHTHALEN-1(2H)-YLIDENE)-3,7-DIMETHYLOCTA-2, 4, 6-TRIENOIC ACID

(71) Applicants: Ulrich ABEL, Bad Homburg (DE); József RÉPÁSI, Érd (HU); András SZABÓ, Budapest (HU); Cserépi Stefánia SZÜCSNÉ, Budapest (HU); Ádám BOR, Budapest (HU); Brickell Biotech, Inc., Boulder, CO (US)

(72) Inventors: Ulrich Abel, Bad Homburg (DE); József Répási, Erd (HU); András Szabó, Budapest (HU); Cserépi Stefánia Szücsné, Budapest (HU); Ádám Bor, Budapest (HU)

(21) Appl. No.: 16/084,905

(22) PCT Filed: Mar. 15, 2017
(86) PCT No.: PCT/EP2017/056125

§ 371 Date: Sep. 13, 2018

(30) Foreign Application Priority Data
Mar. 15, 2016 (EP) 16000647.4

(51) Int. Cl. C07C 51/353; C07C 51/43; C07C 51/09; C07C 29/147; C07C 45/30; C07C 67/307; C07C 67/343

(52) U.S. Cl. CPC C07C 51/353; C07C 51/43; C07C 51/09; C07C 57/50; C07C 45/30; C07C 67/307; C07C 67/343; C07C 29/147

(57) ABSTRACT

This invention relates to a novel method for the synthesis of (2E,4E,6Z,8E)-8-(3,4-dihydronaphthalen-1(2H)-ylidene)-3,7-dimethylocta-2,4,6-trienoic acid. In particular, the invention relates to several improvements in several individual steps of the multi-step synthesis scheme

### FIELD OF THE INVENTION

This invention relates to a novel method for the synthesis of (2E,4E,6Z,8E)-8-(3,4-dihydronaphthalen-1(2H)-ylidene)-3,7-dimethylocta-2,4,6-trienoic acid. In particular, the invention relates to several improvements in several individual steps of the multi-step synthesis scheme.

### BACKGROUND OF THE INVENTION

(2E,4E,6Z,8E)-8-(3,4-dihydronaphthalen-1(2H)-ylidene)-3,7-dimethylocta-2,4,6-trienoic acid (=MRZ-20321) is a known drug substance with RXR agonistic activity.

An initial chemical synthesis to produce milligram quantities of MRZ-20321 was first published by Muccio et al. (1998) (see FIG. 2, route A, steps 3a, 3b, 3c and FIG. 3, route A). The transformation of commercial bromide 2 into acid 5 was carried out in a one-pot three step procedure without isolation of transient intermediates 2B and spiro ester 4. In step 3a, a Reformatsky reaction between 1-tetralone and ethyl 4-bromo-3-methyl-2-butenoate (1:1 mixture of E-2 and Z-2) in dioxane directly provided the crystalline acid Z-5 as a single isomer in 86% yield. Acid Z-5 was reduced with LAH in THF as solvent. The crude alcohol was purified by flash chromatography leading to a 1:1 mixture of alcohols E-7 and Z-7 in 67% yield. The alcohol mixture was further oxidized using a twenty fold excess of MnO₂ in dichloromethane resulting in a crude mixture of aldehydes Z-8 and E-8 and unreacted starting material. The individual isomers were isolated by flash chromatography to give Z-8 (28% yield) and E-8 (25% yield). A Horner-Emmons condensation between pure aldehyde Z-8 and triethyl phosphonosenecioate (1:1 mixture of E-3 and Z-3) in THF and HMPA as solvents provided ester 9 as a 2:1 mixture of 2E-9 and 2Z-9 plus further E/Z isomers in 79% crude yield (FIG. 4, step 6). Ester isomers were separated by HPLC with undisclosed recovery. The separated pure ester 2E-9 was then hydrolyzed under basic conditions to give MRZ-20321.

While satisfactory for a small scale synthesis, this methodology is not amenable for synthesis of multigram quantities. The Reformatzky reaction requires rather tedious handling of heterogeneous mixtures. Even though acid Z-5 is isolated as a crystalline pure isomer, it completely isomerizes in the subsequent reduction step to a mixture of alcohols. The oxidation of the alcohol mixture E-7/Z-7 to the aldehyde mixture E-8/Z-8 required the use of large 20-fold excess of MnO₂ and molecular sieves. On a 100 g scale for example, separation of the product would require washing the MnO₂ with about 15 l of solvent, and during this process, a considerable amount of aldehyde 8 would decompose. At this scale, the yield of the aldehyde 8 is expected to be considerably lower. Purification of the pure desired aldehyde Z-8 by chromatography would become extremely tedious at larger scale. The coupling step 6 yields again a mixture of 2E-9 and 2Z-9 requiring isolation by HPLC which is not easily scalable. Moreover one third of the starting aldehyde Z-8 is lost due to isomerization to the undesired di-Z ester.

The limitations described above apparently prompted Muccio and co-workers to develop an alternative synthetic methodology more amenable for a synthesis of MRZ-20321 on a 100 g scale (Atigadda et al., 2003). The first step also involved a Reformatsky reaction between 1-tetralone and commercial ethyl 4-bromo-3-methyl-2-butenoate (1:1 mixture of E-2 and Z-2) in the presence of Zn and Cu(OAc)₂ in THF (steps 3a and 3b in FIG. 2, route A). However, conditions were used to favour the formation of the intermediate lactone 4 which was isolated in 69% yield (100 g scale). Another major change was the controlled reduction of lactone 4 by DIBAH (step 3d) in THF as a solvent at −78° C., followed by in-situ ring opening and elimination, to provide the aldehyde 8 as a 5:1 mixture of Z-8 and E-8 in a combined yield of 75%. Aldehyde Z-8 was readily separated by chromatography using flash silica. Triethyl phosphonosenecioate (1:1 mixture of E-3 and Z-3) was used to olefinate Z-8 under modified Horner-Emmons conditions to produce the ester 9 (FIG. 4). Under these conditions, the use of excess HMPA and THF as a solvent resulted in a 9:1 mixture of 2E-9 and 2Z-9, and the desired ester 2E-9 was obtained by selective crystallization from ether in 66% yield. Pure 2E-9 was finally hydrolyzed under basic conditions to give the acid MRZ-20321 (yield 78%).

While satisfactory for a 100 g scale synthesis, this method is still not suitable for a synthesis of kilogram quantities in the kilolab, in a pilot plant or for an industrial synthesis. Since the route was found to not be scalable (data not shown), five to seven consecutive batches had to be synthesized to reach a final 500 g quantity. The initial Reformatsky reaction (step 3a) was found to be highly critical. Starting the reaction turned out to be very difficult and poorly reproducible. Test reactions using various methods for Zn activation (Zn—Cu couple, activation with 1,2-dibromoethane) did not lead to a reliable reaction start. Once the Zn insertion step was initiated, it proceeded in a highly exothermic manner and extensive cooling was required to keep it under control. Such “runaway” reactions bear a not controllable hazardous potential and can obviously not be used in larger reactors and therefore need to be replaced. It also was found that in some cases, a reaction start could not be initiated at all for yet unknown reasons and complete batches were lost. Zinc is used in a large excess and needs to be removed from the mixture during workup. The heavy metal copper is used in large quantities and would require careful control and limitation of elemental impurities in a manufacturing process. The direct reduction of lactone 4 (step 3d) to aldehyde 8 requires cryogenic temperatures as low as −78° C. While this is technically doable at a kilolab scale, it is not ideal for an industrial process due to long cooling times and high energy consumption. It is mandatory to quench the reaction mixture also at cryogenic temperatures in order to yield favourable isomeric ratios of Z-8 versus unwanted E-8. In general, the resulting isomeric ratio of aldehyde 8 is highly critical and variable. In ideal cases, the isomer ratio was found to be 5:1 in favour of the desired Z-8. In many other cases, however, a complete isomerization to a 1:1 mixture occurred during the reaction. The resulting mixture needs to be separated by flash chromatography and a significant amount of intermediate is lost due to decomposition and additional isomerization of the rather labile aldehyde. In an industrial setup, such chromatographic separations should be avoided whenever possible (particularly in early synthesis steps) since such separations are hard to scale, time consuming and costly. The Homer Emmons condensation of aldehyde Z-8 and phosphonate 3 requires HMPA as a solvent which is a known carcinogen. Moreover the E/Z ratio of the resulting ester 9 is varying by far and difficult to control. Ratios were found to vary from 2:1 to 9:1. Even though the wanted isomer can be purified by crystallization, the reaction can be considered to be critical and the method is not well suited for an industrial process.

Furthermore, for the bromination step 1, CCl₄ was used as a solvent. This material has been banned for industrial use since it is a severe killer of atmospheric ozone and therefore needs to be replaced for an industrial process.

Even when reaction conditions were carefully kept constant for step 3a, the reaction would regularly fail completely for unknown reasons.

In summary, the previously known synthesis routes for MRZ-20321 were found to be suitable to produce material in a scale up to 100 g. However, the synthesis comprises several critical steps with varying outcome regarding product yield and isomeric ratios. The reduction step requires cryogenic conditions; other steps make use of obsolete toxic or hazardous solvents and reagents. Many synthetic steps result in E/Z mixtures of intermediates which require tedious purification e.g. by flash chromatography. Even when reaction conditions were carefully kept constant for some steps, the reaction would regularly fail completely for unknown reasons. Therefore, the state of the art process is not suitable to support late stage preclinical and clinical development with kg quantities of high quality material. It is not suited for a later manufacture of market API. As a consequence, synthesis conditions of many steps underwent major optimization. Main optimization goals were thereby the replacement of toxic and hazardous reagents; improvement of product selectivity in each step; purification solely by crystallization and by distillation avoiding any chromatographic purification.

Thus, despite the progress that has been made in the past with respect to the synthesis of MRZ-20321, there is still a strong demand to further improve the overall synthesis scheme in order to be able to synthesize MRZ-20321 on a kilogram scale in a safe, cost- and resource-efficient and reliable manner. To date, such aspects have not been addressed satisfactorily.

### OBJECTS OF THE INVENTION

It was an object of the invention to provide improvements to the synthesis of (2E,4E,6Z,8E)-8-(3,4-dihydronaphthalen-1(2H)-ylidene)-3,7-dimethylocta-2,4,6-trienoic acid so that synthesis on a kilogram scale would be possible. The solution to this problem, i.e. the identification of modifications to the synthesis scheme that had been used so far for the synthesis of MRZ-20321, were neither taught nor suggested by the prior art.

### SUMMARY OF THE INVENTION

The present invention is based on the surprising finding that a set of modifications to the synthesis scheme that had been used so far for the synthesis of MRZ-20321 resulted in a simple, reliable, highly efficient synthesis and scalable process that permits the production of MRZ-20321 in quantities large enough for the preclinical and clinical development and for the commercial production of the drug substance.

Thus, the present invention relates in a first aspect to a method for the synthesis of MRZ-20321 comprising one or more of the steps of:
(a) synthesizing 2 as a mixture of isomers E-2/Z-2 by performing a bromination of 1 in a solvent selected from benzotrifluoride and 1,3-bis(trifluoromethyl)benzene, particularly benzotrifluoride;
(b) lithiating 1;
(c) adding tetralone to lithiated 1 to form Z-5;
(d) synthesizing Z-7 starting from Z-5, wherein said method comprises the step of synthesizing the methyl ester Z-6;
(e) reducing Z-6 to obtain Z-7;
(f) oxidizing Z-7 with stabilized 2-iodoxybenzoic acid (SIBX);
(g) reacting Z-8 with 3 (as mixture of isomers E-3/Z-3) in the presence of a lithium dialkylamide, particularly lithium diisopropylamide or lithium diethylamide, particularly lithium diisopropylamide; and/or
(h) recrystallizing MRZ-20321 from isopropanol or from n-heptane or from mixtures of n-heptane and 2-methyl tetrahydrofuran.

In a second aspect, the present invention relates to a method for the synthesis of MRZ-20321 comprising the step of synthesizing 2 (as mixture of E/Z isomers) by performing a bromination of 1 in a solvent selected from benzotrifluoride and 1,3-bis(trifluoromethyl)benzene, particularly benzotrifluoride.

In a third aspect the present invention relates to a method for the synthesis of MRZ-20321 comprising the step of lithiating 1.

In a fourth aspect the present invention relates to a method for the synthesis of MRZ-20321 comprising the step of synthesizing the methyl ester Z-6.

In a fifth aspect the present invention relates to a method for the synthesis of MRZ-20321 comprising the step of reducing Z-6 to obtain Z-7.

In a sixth aspect the present invention relates to a method for the synthesis of MRZ-20321 comprising the step of oxidizing Z-7 with stabilized 2-iodoxybenzoic acid (SIBX).

In a seventh aspect the present invention relates to a method for the synthesis of MRZ-20321 comprising the step of reacting Z-8 with 3 (as mixture of isomers E-3/Z-3) in the presence of a lithium dialkylamide, particularly lithium diisopropylamide or lithium diethylamide, particularly lithium diisopropylamide.

In an eighth aspect the present invention relates to a method for the synthesis of MRZ-20321 comprising the step of recrystallizing MRZ-20321 from isopropanol or from n-heptane or from mixtures of n-heptane and 2-methyl tetrahydrofuran.

In a further aspect the present invention relates to a method for the synthesis of 2 (mixture of E/Z isomers) comprising the step of performing a bromination of 1 in a solvent selected from benzotrifluoride and 1,3-bis(trifluoromethyl)benzene, particularly benzotrifluoride.

In a further aspect the present invention relates to a composition comprising 1, a bromination reagent and a solvent selected from benzotrifluoride and 1,3-bis(trifluoromethyl)benzene, particularly benzotrifluoride.

In a further aspect the present invention relates to a method for the synthesis of Z-5 comprising the step of lithiating 1.

In a further aspect the present invention relates to a composition comprising 1, and a lithiating reagent.

In a further aspect the present invention relates to a composition comprising lithiated 1 and tetralone.

In a further aspect the present invention relates to a method for the synthesis of Z-7 starting from Z-5, wherein said method comprises the step of synthesizing the methyl ester Z-6.

In a further aspect the present invention relates to a composition comprising Z-5 and a methylation reagent.

In a further aspect the present invention relates to a composition comprising Z-6 and a reducing reagent.

In a further aspect the present invention relates to a method for the synthesis of Z-8 comprising the step of oxidizing Z-7 with stabilized 2-iodoxybenzoic acid (SIBX).

In a further aspect the present invention relates to a composition comprising Z-7 and stabilized 2-iodoxybenzoic acid (SIBX).

In a further aspect the present invention relates to a method for the synthesis of 2E-9 comprising the step of reacting Z-8 with E-3/Z-3 in the presence of a lithium dialkylamide, particularly lithium diisopropylamide or lithium diethylamide, particularly lithium diisopropylamide.

In a further aspect the present invention relates to a composition comprising Z-8, E-3/Z-3 and a lithium dialkylamide, particularly lithium diisopropylamide or lithium diethylamide, particularly lithium diisopropylamide.

In a further aspect the present invention relates to a method for the purification of MRZ-20321 comprising the step of recrystallizing MRZ-20321 from isopropanol or from n-heptane or from mixtures of n-heptane and 2-methyl tetrahydrofuran.

### FIGURES

The drawings described herein are for illustration purposes only and are not intended to limit the scope of the present disclosure in any way. Embodiments of the present invention will become more fully understood from the detailed description and the accompanying drawings.

FIG. 1 shows the synthesis scheme for phosphonate 3.

FIG. 2 shows the synthesis scheme towards acid Z-5 and aldehyde Z-8; Muccio et al., 1998: 4 was not isolated and hydrolyzed directly in step 3c; Atigadda et al., 2003: 4 was isolated and reduced in step 3d, resulting in 1:5 mixture of Z-8 and E-8, Z-8 separated by flash.

FIG. 3 shows the synthesis scheme towards aldehyde Z-8; step 4: Muccio et al., 1998: 1:1 mixture of E-7 and Z-7, not separated and directly used in step 5, resulting in 1:1 mixture of E-8 and Z-8; Z-8 separated by flash.

FIG. 4 shows the synthesis scheme for the final steps towards MRZ-20321; step 6: Muccio et al., 1998: 2:1 mixture of 2E-9 and 2Z-9, 2E-9 separated by HPLC; Atigadda et al., 2003: 9:1 mixture of 2E-9 and 2Z-9, 2E-9 separated by crystallization.

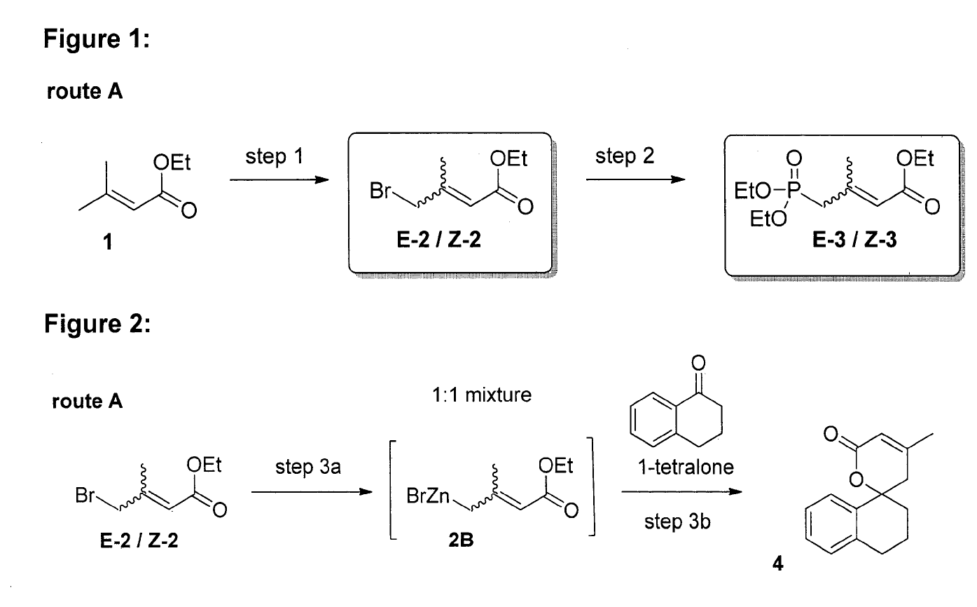

### DETAILED DESCRIPTION OF THE INVENTION

The present invention may be understood more readily by reference to the following detailed description of the invention and the examples included therein.

Thus, the present invention relates in a first aspect to a method for the synthesis of MRZ-20321 comprising one or more of the steps of:
(a) synthesizing E-2/Z-2 by performing a bromination of 1 in a solvent selected from benzotrifluoride and 1,3-bis(trifluoromethyl)benzene, particularly benzotrifluoride;
(b) lithiating 1;
(c) adding tetralone to lithiated 1;
(d) synthesizing Z-7 starting from Z-5, wherein said method comprises the step of synthesizing the methyl ester Z-6;
(e) reducing Z-6 to obtain Z-7;
(f) oxidizing Z-7 with stabilized 2-iodoxybenzoic acid (SIBX);
(g) reacting Z-8 with E-3/Z-3 in the presence of a lithium dialkylamide, particularly lithium diisopropylamide or lithium diethylamide, particularly lithium diisopropylamide; and/or
(h) recrystallizing MRZ-20321 from isopropanol or from n-heptane or from mixtures of n-heptane and 2-methyl tetrahydrofuran.

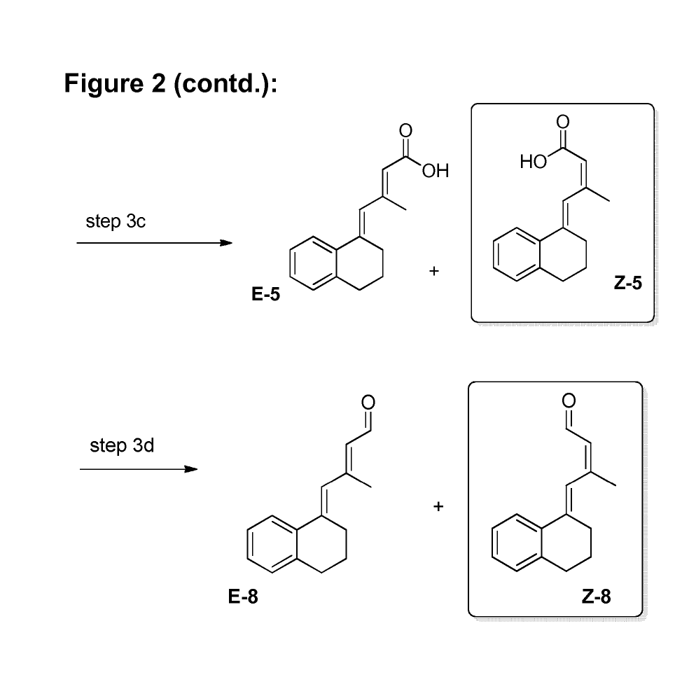

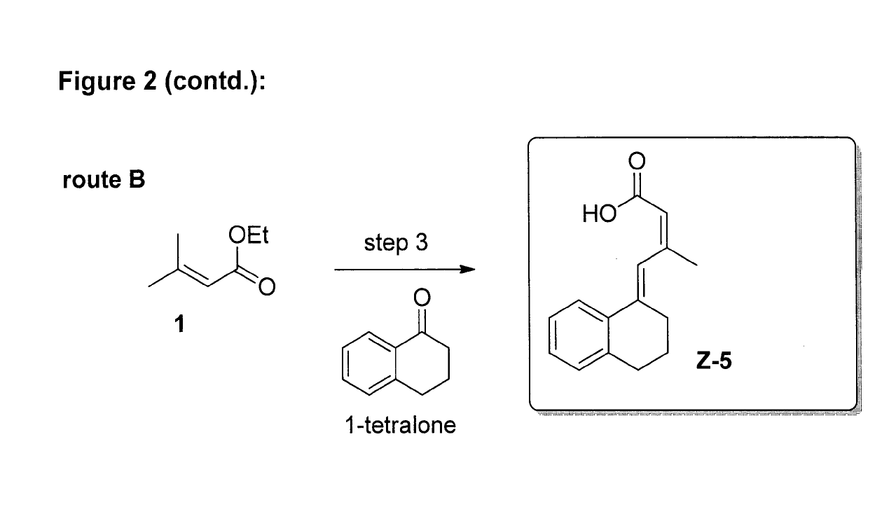

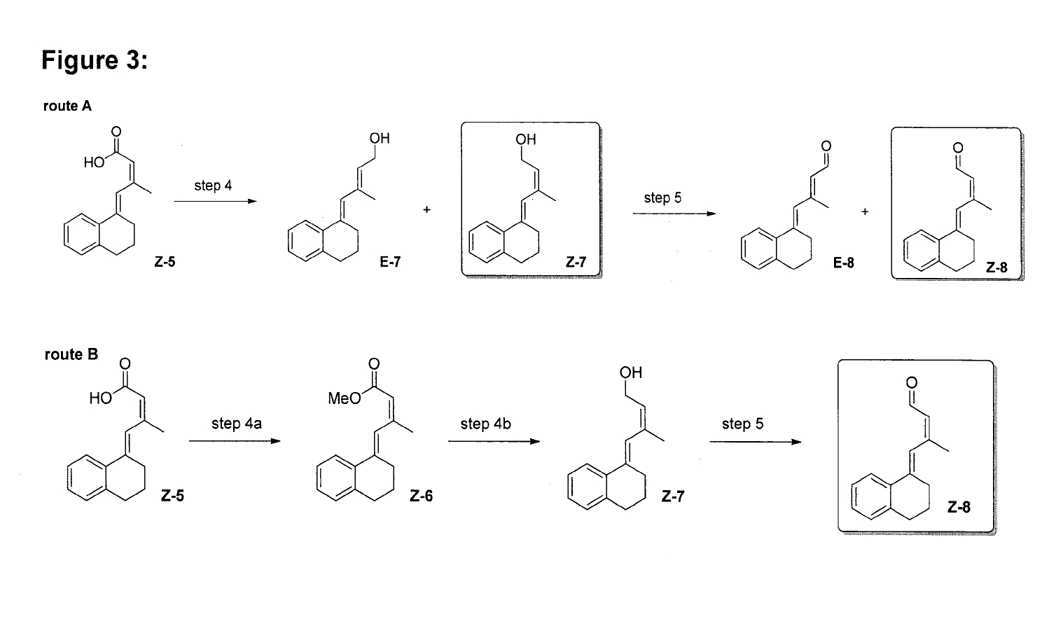

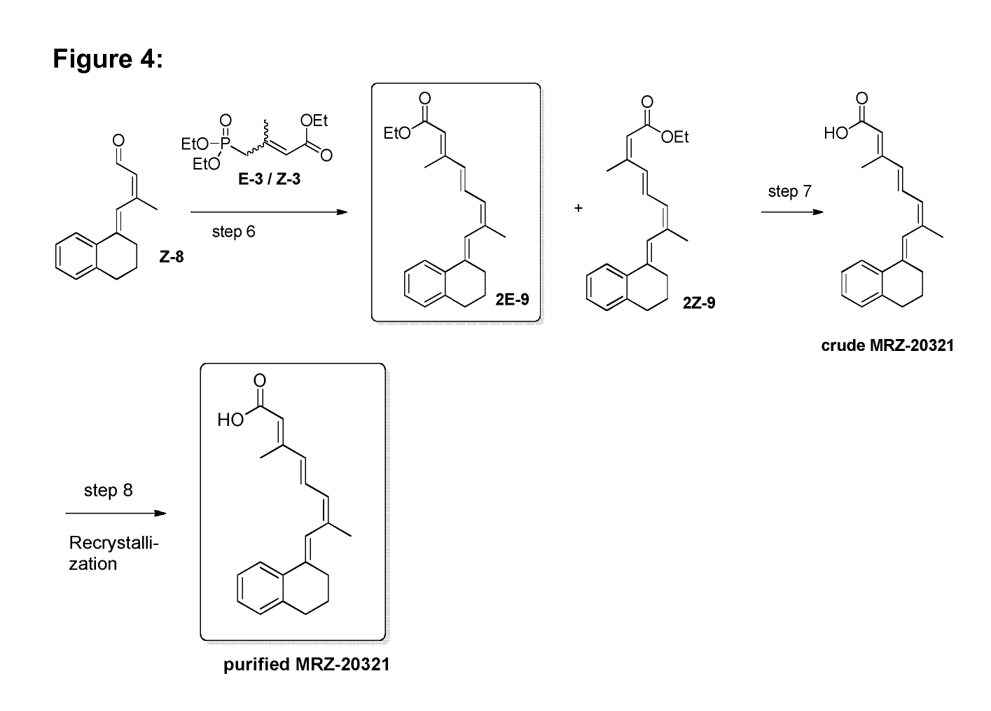

The optimized synthesis process is in part following the route previously presented by Muccio's group. One major improvement was achieved by replacing the critical and unreliable Reformatzky sequence (steps 1, 3a, 3b, 3c) by one single step 3 via the direct lithiation of dimethyl crotonate 1 (FIG. 2, route B). Thereby, synthesis of acid Z-5 was significantly shortened, and criticality was reduced by far. In the bromination step 1a, obsolete CCl₄ was replaced by less toxic and harmful benzotrifluoride. A major improvement was replacing the reduction step 4 by a two-step sequence via methyl ester Z-6 (steps 4a and 4b). This sequence turned out to be highly reproducible and to proceed without any isomerization of products, thus avoiding extensive purification and loss of material. In the oxidation step 5, explosive IBX was replaced by sIBX (“stabilized” IBX) which can be used safely on a large scale. A method was discovered for removing the stabilizers during workup which was prerequisite for use of sIBX. Moreover, coupling of aldehyde 8 with phosphonate 3 was optimized. It was discovered that the choice of base and temperature has a large influence on product selectivity. It was unexpectedly found that when LDA was used as a base for deprotonation of phosphonate 3, the coupling product 2E-9 was synthesized in a 9:1 selectivity towards 2Z-9 in the reaction mixture. Crystallization from isopropanol or from 2-methyl tetrahydrofuran yielded desired ester 2E-9 in 99% isomeric purity and a good yield of 79%. Most strikingly, the outcome was independent of the isomeric ratio of starting material 3. Using either pure E-3 or a 1:1 mixture of E-3 and Z-3 evenly resulted in formation of pure isomer 2E-9.

As a consequence, all previously critical steps are now highly controllable. Isomeric product ratios are much more favorable, purifications are solely based on crystallization without the need for chromatographic purifications. The outcome of every step is highly predictable and reproducible and therefore non-critical. The feasibility of the improved process was recently proven by the synthesis of a 2.4 kg demo batch of MRZ-20321. Phosphonate 3 and alcohol Z-8 were successfully produced on a 10 kg pilot plant scale. As a result, the synthesis costs per gram of RZ-20321 could be significantly reduced as compared to costs following known routes.

TABLE 1
Improvements by synthesis step:

| | characteristics and | |
|---|---|---|
| | shortcomings of previous | advantages of improved |
| Step | procedures | procedure |
| step 1 | Bromination with NBS using | CCl₄ is replaced by less toxic |
| (bromination) | CCl₄ as solvent. CCl₄ is | benzotrifluoride. |
| | banned by the Kyoto protocol | |
| | and obsolete for industrial | |
| | production (harmful to the | |
| | environment). It is extremely | |
| | toxic. | |
| step 3a-3d | Muccio 2003 route (3a, 3b, 3d) | route B step 3 |
| (Reformatzky) | In step 3a, Zn is activated | Reformatzky is replaced by direct |
| | using toxic heavy metal Cu. | lithiation step. Critical formation |
| | Toxic benzene is used. | of Zn-organyl completely |
| | Lactone reduction step 3d | avoided. Multistep sequence |
| | yields a 4:1 mixture of Z-8 and | replaced by a single reliable |
| | unwanted E-8 which requires | reaction step. |
| | flash chromatography | |
| | Muccio 1998 route (3a, 3b in | |
| | situ, 3c): | |
| | Zn activation is complicated | |
| | and critical. | |
| step 4 | Direct reduction of Z-5 requires | Replaced by two step procedure |
| (reduction) | cryogenic conditions; | (steps 4a and 4b) leading to full |
| | isomerization of product Z-7 is | control over product selectivity |
| | not controllable. | towards Z-7. As a consequence, |
| | | the subsequence oxidation also |
| | | leads to a pure isomer Z-8 thus |
| | | avoiding any chromatographic |
| | | purification. No extreme |
| | | cryogenic conditions required. |
| step 5 | Explosive IBX is used, only | IBX replaces by stabilized sIBX. |
| (oxidation) | 100 g scale synthesis possible. | Stabilizers could be removed |
| | | during workup. Process is safe |
| | | and scalable. |
| step 6 | Varying results regarding | High product selectivity, |
| (coupling) | product selectivity | reproducible process, leads to |
| | | pure 2E-9 independent from |
| | | isomeric purity of starting |
| | | phosphonate 3. |

In a second aspect, the present invention relates to a method for the synthesis of MRZ-20321 comprising the step of synthesizing E-2/Z-2 by performing a bromination of 1 in a solvent selected from benzotrifluoride and 1,3-bis(trifluoromethyl)benzene, particularly benzotrifluoride.

In a particular embodiment, said bromination is performed with N-bromosuccinimide.

In a particular embodiment, said bromination is performed by using a radical initiator selected from azobisisobutyronitrile, and dibenzoyl peroxide, particularly azobisisobutyronitrile.

In a third aspect the present invention relates to a method for the synthesis of MRZ-20321 comprising the step of lithiating 1.

Direct lithiations of similar nature had already been reported in the prior art (Dugger et al., 1980; Ballester et al., 1989). However, despite being known since long, it had so far not been recognized that this approach can be employed with surprisingly high efficacy for the synthesis of compound Z-5 as precursor for MRZ-20321.

In a particular embodiment, said lithiating step is performed by using a lithiating reagent selected from a lithium dialkylamide, particularly lithium diisopropylamide or lithium diethylamide; a lithium, sodium or potassium salt of bis(trimethylsilyl)amide (HMDS), particularly lithium bis(trimethylsilyl)amide; and lithium tetramethylpiperidine.

In a particular embodiment, said method further comprises the step of adding tetralone to the lithiated 1.

In a fourth aspect the present invention relates to a method for the synthesis of MRZ-20321 comprising the step of synthesizing the methyl ester Z-6.

In a particular embodiment, said step comprises reacting Z-5 with a methylation reagent.

In a particular embodiment, said methylation reagent comprises methyl iodide and a base, particularly a base selected from potassium carbonate; sodium carbonate; a tertiary amine, particularly selected from N,N-diisopropylethylamine and triethylamine; and DBU.

In a fifth aspect the present invention relates to a method for the synthesis of RZ-20321 comprising the step of reducing Z-6 to obtain Z-7.

In a particular embodiment, said step of reducing Z-6 is performed using a reducing reagent selected from an alkyl aluminium hydride, particularly selected from lithium aluminium hydride and DIBAH (diisobutyl aluminium hydride), particularly lithium aluminium hydride; an alkoxy aluminium metal hydride, particularly selected from Red-Al (sodium bis(2-methoxyethoxy)-aluminium hydride) and lithium tri-tert-butoxyaluminium hydride; an alkyl borohydride, particularly selected from 9-BBN, NaBH₄; LiBH₄; borane dimethyl sulfide complex; and borane THF complex; and an alkoxy borohydride, particularly sodium triacetoxy borohydride.

In a particular embodiment, said method further comprises the step of using potassium sodium tartrate in the work-up procedure after the reducing reaction.

In a particular embodiment, said method further comprises the step of recrystallizing the raw product Z-7.

In a sixth aspect the present invention relates to a method for the synthesis of RZ-20321 comprising the step of oxidizing Z-7 with stabilized 2-iodoxybenzoic acid (SIBX).

In a particular embodiment, said method further comprises the removal of isophthalic acid, iodosobenzoic acid and unreacted SIBX.

In a particular embodiment, said method further comprises the removal of benzoic acid.

In a particular embodiment, said method further comprises the step of recrystallizing the raw product obtained in said step of oxidizing Z-7.

In a seventh aspect the present invention relates to a method for the synthesis of MRZ-20321 comprising the step of reacting Z-8 with E-3/Z-3 in the presence of a lithium dialkylamide, particularly lithium diisopropylamide or lithium diethylamide, particularly lithium diisopropylamide.

In a particular embodiment, said step of reacting Z-8 with E-3/Z-3 is performed at a temperature between −50° C. and −30° C.

In an eighth aspect the present invention relates to a method for the synthesis of MRZ-20321 comprising the step of recrystallizing MRZ-20321 from isopropanol or from n-heptane or from mixtures of n-heptane and 2-methyl tetrahydrofuran.

In a further aspect the present invention relates to a method for the synthesis of E-2/Z-2 comprising the step of performing a bromination of 1 in a solvent selected from benzotrifluoride and 1,3-bis(trifluoromethyl)benzene, particularly benzotrifluoride.

In a particular embodiment, said bromination is performed with N-bromosuccinimide.

In a particular embodiment, said bromination is performed by using a radical initiator selected from azobisisobutyronitrile, and dibenzoyl peroxide, particularly azobisisobutyronitrile.

In a further aspect the present invention relates to a composition comprising 1, a bromination reagent and a solvent selected from benzotrifluoride and 1,3-bis(trifluoromethyl)benzene, particularly benzotrifluoride.

In a particular embodiment, said bromination reagent comprises N-bromosuccinimide.

In a particular embodiment, said bromination reagent further comprises a radical initiator selected from azobisisobutyronitrile, and dibenzoyl peroxide, particularly azobisisobutyronitrile.

In a further aspect the present invention relates to a method for the synthesis of Z-5 comprising the step of lithiating 1.

In a particular embodiment, said lithiating step is performed by using a lithiating reagent, particularly a lithiating reagent selected from a lithium dialkylamide, particularly lithium diisopropylamide or lithium diethylamide; a lithium, sodium or potassium salt of bis(trimethylsilyl)amide (HMDS), particularly lithium bis(trimethylsilyl)amide; and lithium tetramethylpiperidine.

In a particular embodiment, said method further comprises the step of adding tetralone to the lithiated 1.

In a further aspect the present invention relates to a composition comprising 1, and a lithiating reagent.

In a particular embodiment, said lithiating reagent is a lithiating reagent selected from a lithium dialkylamide, particularly lithium diisopropylamide or lithium diethylamide; a lithium, sodium or potassium salt of bis(trimethylsilyl)amide (HMDS), particularly lithium bis(trimethylsilyl)amide; and lithium tetramethylpiperidine.

In a further aspect the present invention relates to a composition comprising lithiated 1 and tetralone.

In a further aspect the present invention relates to a method for the synthesis of Z-7 starting from Z-5, wherein said method comprises the step of synthesizing the methyl ester Z-6.

In a particular embodiment, said step comprises reacting Z-5 with a methylation reagent.

In a particular embodiment, said methylation reagent comprises methyl iodide and a base, particularly a base selected from potassium carbonate; sodium carbonate; a tertiary amine, particularly selected from N,N-diisopropylethylamine and triethylamine; and DBU.

In a particular embodiment, said method further comprises the step of reducing Z-6 to obtain Z-7.

In a particular embodiment, said step of reducing Z-6 is performed using a reducing reagent selected from an alkyl aluminum hydride, particularly selected from lithium aluminium hydride and DIBAH (diisobutyl aluminium hydride), particularly lithium aluminium hydride; an alkoxy aluminum metal hydride, particularly selected from Red-Al (sodium bis(2-methoxyethoxy)-aluminium hydride) and lithium tri-tert-butoxyaluminium hydride; an alkyl borohydride, particularly selected from 9-BBN, NaBH4; LiBH4; borane dimethyl sulfide complex; and borane THF complex; and an alkoxy borohydride, particularly sodium triacetoxy borohydride.

In a particular embodiment, said method further comprises the step of using potassium sodium tartrate in the work-up procedure after the reducing reaction.

In a particular embodiment, said method further comprises the step of recrystallizing the raw product Z-7.

In a further aspect the present invention relates to a composition comprising Z-5 and a methylation reagent.

In a particular embodiment, said alkylating reagent comprises methyl iodide and a base, particularly a base selected from potassium carbonate; sodium carbonate; a tertiary amine, particularly selected from N,N-diisopropylethylamine and triethylamine; and DBU.

In a further aspect the present invention relates to a composition comprising Z-6 and a reducing reagent.

In a particular embodiment, said reducing reagent comprises a reducing reagent selected from an alkyl aluminum hydride, particularly selected from lithium aluminium hydride and DIBAH (diisobutyl aluminium hydride), particularly lithium aluminium hydride; an alkoxy aluminum metal hydride, particularly selected from Red-Al (sodium bis(2-methoxyethoxy)-aluminium hydride) and lithium tri-tert-butoxyaluminium hydride; an alkyl borohydride, particularly selected from 9-BBN, NaBH4; LiBH4; borane dimethyl sulfide complex; and borane THF complex; and an alkoxy borohydride, particularly sodium triacetoxy borohydride.

In a further aspect the present invention relates to a method for the synthesis of Z-8 comprising the step of oxidizing Z-7 with stabilized 2-iodoxybenzoic acid (SIBX).

In a particular embodiment, said method further comprises the removal of isophthalic acid, iodosobenzoic acid and unreacted SIBX.

In a further embodiment, said method further comprises the removal of benzoic acid.

In a particular embodiment, said method further comprises the step of recrystallizing the raw product obtained in said step of oxidizing Z-7.

In a further aspect the present invention relates to a composition comprising Z-7 and stabilized 2-iodoxybenzoic acid (SIBX).

In a further aspect the present invention relates to a method for the synthesis of 2E-9 comprising the step of reacting Z-8 with E-3/Z-3 in the presence of a lithium dialkylamide, particularly lithium diisopropylamide or lithium diethylamide, particularly lithium diisopropylamide.

In a particular embodiment, said step of reacting Z-8 with E-3/Z-3 is performed at a temperature between −50° C. and −30° C.

In a further aspect the present invention relates to a composition comprising Z-8, E-3/Z-3 and a lithium dialkylamide, particularly lithium diisopropylamide or lithium diethylamide, particularly lithium diisopropylamide.

In a further aspect the present invention relates to a method for the purification of MRZ-20321 comprising the step of recrystallizing MRZ-20321 from isopropanol or from n-heptane or from mixtures of n-heptane and 2-methyl tetrahydrofuran.

### REFERENCES

1. Muccio, D. D.; Brouillette, W. J.; Breitman, T. R.; Taimi, M.; Emanuel, P. D.; Zhang, X.-k.; Chen, G.-q.; Sani, B. P.; Venepally, P.; Reddy, L., Conformationally defined retinoic acid analogues. 4. Potential new agents for acute promyelocytic and juvenile myelomonocytic leukemias. Journal of medicinal chemistry 1998, 41 (10), 1679-1687.
2. Atigadda, V. R.; Vines, K. K.; Grubbs, C. J.; Hill, D. L.; Beenken, S. L.; Bland, K. I.; Brouillette, W. J.; Muccio, D. D., Conformationally Defined Retinoic Acid Analogues. 5. Large-Scale Synthesis and Mammary Cancer Chemopreventive Activity for (2E,4E,6 Z, 8E)-8-(3′,4′-Dihydro-1′(2′H)-naphthalen-1′-ylidene)-3,7-dimethyl-2,4,6-octatrienoic Acid (9cUAB30). Journal of medicinal chemistry 2003, 46 (17), 3766-3769.
3. Dugger, R. W.; and Heathcock, C. H., A General Synthesis of 5,6-Dihydro-a-pyrones. J. Org. Chem. 1980, 45, 1181-1185.
4. Ballester, P., et al. (1989). “Unsaturated carboxylic acid dienolates. Reaction with substituted cyclohexanones and unsubstituted cycloalkanones. Regio- and stereo-selectivity.” Journal of the Chemical Society, Perkin Transactions 1(1).

### EXAMPLES

### Example 1: Synthesis of E-2/Z-2 (Route A, Step 1)

A 35 l Hastelloy autoclave was charged with 9.6 kg of benzotrifluoride (BTF), 2.0 kg (1.0 eq, 15.6 mol) of ethyl 3,3-dimethylacrylate and 11.6 g (0.045 eq, 0.7 mol) of azobisisobutyronitrile (AIBN, as a radical initiator). The solution was heated to 75° C. and to the solution was added in seven portions at 80-100° C. 2.22 kg (0.8 eq, 12.5 mol) of N-bromosuccinimide. The reaction mixture was stirred further 2 h at 85-95° C. The reaction mixture was cooled down to 15-20° C. The solid succinimide was filtrated off and washed with 3 kg benzotrifluoride. The combined filtrates were evaporated to dryness under diminished pressure at max. 60° C. The crude product was purified by vacuum distillation at 0.4-0.8 mbar. Yield of E-2/Z-2 was 0.899 kg (28%), purity: 91.5% (4:5 mixture of isomers).

### Example 2: Synthesis of E-3 l Z-3 (Route A, Step 2)

A 2 l three-necked, round bottomed flask equipped with stirrer, oil bath, thermometer was charged with 317 g (1.05 eq, 1.91 mol) of triethyl phosphite and heated to 95-100° C. To the triethyl phosphite was added 378 g (1.0 eq, 1.825 mol) of E-2/Z-2 (ethyl-4-bromo-3-methyl crotonate) at 100-120° C. during 1 h. The evolved ethyl bromide was distilled off. The reaction mixture was stirred for 2 h at 100-120° C. and distilled in vacuum at 0.3-0.8 mbar (140-160° C. oil bath temperature). The product was collected at temperature ranging from 100−120° C. Yield of E-3/Z-3 was 383 g (79%), purity: 94.4% (42.1% cis isomer and 52.3% trans isomer).

### Example 3: Synthesis of Z-5 (Route B, Step 3)

A 35 l Hastelloy autoclave was charged at −5-0° C. under nitrogen atmosphere with 7.68 kg (1.2 eq, 20 mol) of lithium diisopropylamide (28% solution in heptane/THF/ethylbenzene). Then 2.29 kg (1.0 eq, 17.9 mol) of ethyl 3,3-dimethylacrylate (1) in 3.1 kg of THF was added portion-wise and the temperature was kept between −2-5° C. Then 2.60 kg (1.0 eq, 17.8 mol) of α-tetralone in 3.1 kg of THF was added portion wise and the temperature was kept between −2-5° C. The reaction mixture was stirred 30 min at −2-5° C. allowed to warm to 20-25° C. and stirring was continued for 2 h. The reaction mixture was quenched with 30 kg of water at 10-20° C. and the layers were separated. The organic phase was washed with 2×7.4 kg of water. The combined aqueous phase was washed with 3×5.6 kg of MTBE and acidified with 7 kg of 50% diluted hydrochloric acid to pH=1-2 in the presence 16 kg of dichloromethane. The resulting mixture was stirred for 15-20 min and the layers were separated. The aqueous phase was extracted with 5 kg of dichloromethane. The combined organic phase was dried over sodium sulphate (0.3 kg) and evaporated to a volume of 2-3 l. 3.5 kg of toluene were added and the residue of dichloromethane was distilled off under reduced pressure at 40° C. The resulted crystalline slurry (2-3 l) was cooled to 0-5° C., agitated for 1 h, filtered and washed with 0.7 kg of cold toluene. The wet intermediate was dried under reduced pressure at 30-40° C. Yield of Z-5 was 1.36 kg (33%), purity: 99.8 area-%.

### Example 4: Synthesis of Z-6 (Route B, Step 4a)

A 35 l Hastelloy autoclave was charged at room temperature with 1.69 kg (1.0 eq, 7.4 mol) of Z-5, 2.55 kg (2.5 eq, 18.5 mol) of potassium carbonate, 1.57 kg (1.2 eq, 11 mol) of methyl iodide and 4 kg of acetone. The reaction mixture was refluxed for 2 h, then cooled to 20-25° C. and the solid was filtered and washed with 3 l of acetone. The organic solution was evaporated to dryness under diminished pressure. 2×0.4 kg of MTBE was added and the residue of acetone was distilled off under diminished pressure at 40° C. After decantation from some solid KI precipitate the yield of crude Z-6 was 1.6 kg (yield 89%) as yellow oil, purity: 99.9% by HPLC

### Example 5: Synthesis of Z-7 (Route B, Step 4b)

A 35 l Hastelloy autoclave was charged under nitrogen atmosphere with 0.750 kg (1.0 eq, 3.1 mol) of Z-6 and 5.7 kg of MTBE and the mixture was cooled to −40 to −25° C. 0.86 kg (1.1 eq, 3.4 mol) of a LAH solution (lithium aluminium hydride, 15% solution in THF/toluene) was added in a period of 1.5 h at −40-(−23)° C. The resulting reaction mixture was stirred for 1 h at −40 to −25° C. The reaction mixture was quenched with 0.29 kg of methanol at −40 to −20° C. and with 3.4 kg of water at −25 to 5° C. Diluted HCl solution (2:1) was added and the pH was adjusted to 5-6 at 0-5° C. The layers were separated and the water/precipitate phase was extracted with 2×1 kg of MTBE. The combined organic phase was dried over sodium sulphate (0.2 kg), and evaporated to 1-2 l under diminished pressure at max. 40° C. To the residue 3.0 kg of n-Hexane was added at 35-40° C., then cooled to 0-5° C. and agitated for 1-2 h. The formed precipitate was filtered and washed with a cold mixture of 0.7 kg of n-Hexane and 0.15 kg of MTBE. The wet intermediate was dried under diminished pressure at 20-30° C. Yield of Z-7 was 0.53 kg (80%), purity 97.8%.

### Example 6: Synthesis of Z-8 (Route B, Step 5)

A 35 l Hastelloy autoclave was charged with 3.37 kg of SIBX (1.7 eq, active ingredient IBX 4.8 mol) and 7.3 kg of acetone. The suspension was heated to 45-50° C. 0.623 kg (1.0 eq, 2.9 mol) of Z-7 was dissolved in 2.4 kg of acetone and added to the suspension. The reaction mixture was heated to reflux and stirred for 1 h. The reaction mixture was cooled down to 10 to 15° C. and the solid (mixture of Isophthalic acid, benzoic acid, IBA and unreacted IBX) was filtered and washed with 2×1.8 kg of acetone. The combined organic phase was evaporated to dryness under diminished pressure (water bath max. 35° C.). Then 2×0.6 kg of diisopropyl ether was added and the residual acetone was distilled off under diminished pressure at max. 35° C. The resulting solid (mixture of Z-8 and benzoic acid) at 20 to 25° C. was suspended in 4.6 kg diisopropyl ether and washed with 5.28 kg sodium carbonate solution (5% w/w) four times The organic phase was dried over sodium sulphate and evaporated under diminished pressure at max. 35° C. to 1.6-1.8 kg. The solution was cooled to −5 to 0° C. After 2 h the solid precipitate was filtered and washed with cold 0.5 kg diisopropyl ether and dried at 20 to 25° C. under diminished pressure. Yield of Z-8 was 0.452 kg (73%); purity 99.9 area-%.

### Example 7: Synthesis of 2E-9 (Step 6)

A 35 l Hastelloy autoclave was charged with 0.43 kg (1.2 eq, 1.63 mol) of E-3/Z-3, 1.6 kg of THF and the solution was cooled to −40 to −35° C. To the solution was added at −40 to −30° C. 0.64 kg (1.2 eq, 1.66 mol) of a LDA solution (28% solution in heptane/THF ethylbenzene). The reaction mixture was stirred 1 h at −40 to −30° C. A mixture of 0.287 kg (1.0 eq., 1.35 mol) Z-8 and 1.1 kg THF was added to the reaction mixture at −40 to −30° C. The reaction was stirred at −40 to −30° C. and monitored with HPLC. The reaction mixture was quenched with 3.5 kg of water at −40 to −20° C. and the layers were separated. The water phase was extracted with 2.2 kg of MTBE and 2×0.9 kg of MTBE. The combined organic phase was washed with 0.5 kg of brine and dried over sodium sulphate (0.2 kg) and concentrated under diminished pressure at max. 30° C. to 0.57-0.65 kg. The evaporation residue was dissolved in 1.45 kg 2-propanol. The solution was cooled to −20 to −10° C., and agitated for 1-2 h to give a crystalline suspension. The solid was filtered, washed with 0.5 kg cold isopropanol and dried under vacuum at max. 30° C. Yield 65% of 2E-9, purity: 99.1%.

### Example 8: Synthesis of Crude MRZ-20321 (Step 7)

A 35 l Hastelloy autoclave was charged with 0.523 kg (1.0 eq 0.88 mol) of 2E-9 and 8.3 kg of methanol. 0.523 kg of Potassium hydroxide was dissolved in 8.7 kg of deionized water and added to the suspension. The reaction mixture was heated to reflux and agitated for 1.5 h. The reaction mixture was cooled to 0-5° C. and acidified with diluted (1:1) HCl to pH 2-3. The resulting suspension was filtered, washed with 4×2.5 kg water, then with 2×0.7 kg heptanes, and finally with 3×0.5 kg cold 2-propanol. The crude product was dried under vacuum at max. 30° C. Yield of crude MRZ-20321: 0.523 kg (91%), purity 98.3 area-%.

### Example 9: Purification (Step 8)

In a 35 l Hastelloy autoclave 0.655 kg of crude MRZ-20321 was dissolved in 26 kg of 2-propanol at 58-62° C. 10 g of celite was added to solution, and agitated at 58-62° C. for 15-30 min. The suspension was filtered and concentrated under diminished pressure at max. 40° C. to a volume of 6-8 l. The mixture was cooled to 0-5° C. and agitated for 2 h. The resulting suspension was filtered and washed with 0.8 kg of cold 2-propanol. The wet product was dried under vacuum at max. 30° C. Yield of MRZ-20321: 0.569 kg (87%); purity: 99.9 area-%.

### Example 10: Alternative Synthesis of Crude MRZ-20321 (Step 7)

A 100 l glass lined autoclave was charged with: 3.2 kg (1.0 eq 10 mol) of 2E-9 and 24 kg of methanol. 3.0 kg of potassium hydroxide was dissolved in 30 kg of deionized water and added to the suspension. The reaction mixture was heated to reflux and agitated for 3 h. The reaction mixture was cooled to 30-35° C. and the methanol was distilled off under diminished pressure at max. 50° C. The aqueous mixture was diluted with 30 kg of 2-methyl-tetrahydrofurane, and acidified with diluted (1:1) HCl to pH 2-2.5 at 15-20° C. The organic layer was washed with 2×20 kg of demineralised water. The organic layer was pre-filtered and concentrated under diminished pressure at max. 50° C. to a volume of 7-9 l. Then 14 kg of n-heptane was added and the mixture was concentrated under diminished pressure at max. 50° C. to a volume of 7-9 l. The resulting suspension was diluted with 10 kg of n-heptane, heated to 55-60° C. and agitated for 30 min. Then it was cooled to 0-5° C. After 1 h, the solid precipitate was filtered and washed with 6 kg of n-heptane.

### Example 11: Alternative Purification (Step 8)

The wet product (2.6 kg) was dissolved in 23.4 kg of 2-methyl-tetrahydrofuran at 20-25° C. The mixture was pre-filtered and concentrated under diminished pressure at max. 50° C. to a volume of 7-9 l. Then 14 kg of n-heptane was added and concentrated under diminished pressure at max. 50° C. to a volume of 7-9 l. The remained suspension was diluted with 10.4 kg of n-heptane, heated to 55-60° C. and agitated for 30 min. Then it was cooled to 0-5° C. After 1 h, the solid precipitate was filtered and washed with 5 kg of n-heptane. The wet product was dried at 35° C. under diminished pressure. Yield 2.47 kg (93%); purity 99.8 area-% by HPLC.

1. A method for the synthesis of MRZ-20321 comprising one or more of the steps of:
(a) synthesizing E-2/Z-2 by performing a bromination of 1 in a solvent selected from benzotrifluoride and 1,3-bis(trifluoromethyl)benzene, particularly benzotrifluoride;
(b) lithiating 1;
(c) adding tetralone to lithiated 1;
(d) synthesizing Z-7 starting from Z-5, wherein said method comprises the step of synthesizing the methyl ester Z-6;
(e) reducing Z-6 to obtain Z-7;
(f) oxidizing Z-7 with stabilized 2-iodoxybenzoic acid (SIBX);
(g) reacting Z-8 with E-3/Z-3 in the presence of a lithium dialkylamide, particularly lithium diisopropylamide or lithium diethylamide, particularly lithium diisopropylamide; and/or
(h) recrystallizing MRZ-20321 from isopropanol or from n-heptane or from mixtures of n-heptane and 2-methyl tetrahydrofuran.

2. The method of claim 1, wherein step (a) is performed in benzotrifluoride as solvent.

3. The method of claim 1 or 2, wherein said bromination in step (a) is performed with N-bromosuccinimide.

4. The method of claim 3, wherein said bromination is performed by using a radical initiator selected from azobisisobutyronitrile, and dibenzoyl peroxide, particularly azobisisobutyronitrile.

5. The method of any one of claims 1 to 4, wherein said lithiating in step (b) is performed by using a lithiating reagent selected from a lithium dialkylamide, particularly lithium diisopropylamide or lithium diethylamide; a lithium, sodium or potassium salt of bis(trimethylsilyl)amide (HMDS), particularly lithium bis(trimethylsilyl)amide; and lithium tetramethylpiperidine.

6. The method of any one of claims 1 to 5, wherein said step (d) comprises reacting Z-5 with a methylation reagent.

7. The method of claim 6, wherein said methylation reagent comprises methyl iodide and a base, particularly a base selected from potassium carbonate; sodium carbonate; a tertiary amine, particularly selected from N,N-diisopropylethylamine and triethylamine; and DBU.

8. The method of any one of claims 1 to 7, wherein said step (e) is performed using a reducing reagent selected from an alkyl aluminum hydride, particularly selected from lithium aluminium hydride and DIBAH (diisobutyl aluminium hydride), particularly lithium aluminium hydride; an alkoxy aluminum metal hydride, particularly selected from Red-Al (sodium bis(2-methoxyethoxy)-aluminium hydride and lithium tri-tert-butoxyaluminium hydride; an alkyl borohydride, particularly selected from 9-BBN, NaBH₄; LiBH₄; borane dimethyl sulfide complex; and borane THF complex; and an alkoxy borohydride, particularly sodium triacetoxy borohydride.

9. The method of claim 8, wherein said method further comprises the step of using potassium sodium tartrate in the work-up procedure after the reducing reaction.

10. The method of claim 8 or 9, wherein said method further comprises the step of recrystallizing the raw product Z-7.

11. The method of any one of claims 1 to 10, wherein said step (f) further comprises the removal of isophthalic acid, iodosobenzoic acid and unreacted SIBX.

12. The method of claim 11, wherein said step (f) further comprises the removal of benzoic acid.

13. The method of claim 11 or 12, wherein said step (f) further comprises the step of recrystallizing the raw product obtained in said step of oxidizing Z-7.

14. The method of any one of claims 1 to 13, wherein said step (g) of reacting Z-8 with E-3/Z-3 is performed at a temperature between −50° C. and −30° C.
